(19) United States
(12) Patent Application Publication
(10) Pub. No.: US 2022/0259124 A1
(43) Pub. Date: Aug. 18, 2022

(54) PROCESS FOR PREPARING BISPHENOLE A (BPA) IN THE PRESENCE OF HYDROXYACETONE

(71) Applicant: Covestro Intellectual Property GmbH & Co. KG, Leverkusen (DE)

(72) Inventors: Jerico Jayson Uy, Antwerpen (BE); Erik Sluyts, Brasschaat (BE)

(21) Appl. No.: 17/627,938

(22) PCT Filed: Aug. 24, 2020
(86) PCT No.: PCT/EP2020/073592

§ 371 Date: Jan. 18, 2022

(30) Foreign Application Priority Data
Aug. 27, 2019 (EP) 19193680.6

(51) Int. Cl. C07C 37/20; C07C 37/84; C08G 64/06

(52) U.S. Cl. CPC C07C 37/20; C08G 64/06; C07C 37/84

(57) ABSTRACT

The present invention relates to a process for preparing bisphenol A in the presence of hydroxyacetone without poisoning the catalyst system comprising an ion exchange resin catalyst and a sulfur containing cocatalyst, wherein at least part of the sulfur containing cocatalyst is not chemically bound to the ion exchange resin catalyst. Moreover, the present invention provides a process for preparing polycarbonate and a composition comprising bisphenol A and at least one specific impurity which is formed in the production of bisphenol A.

### CROSS REFERENCE TO RELATED APPLICATIONS

This application is a U.S. national stage application, filed under 35 U.S.C. § 371, of International Application No. PCT/EP2020/073592, which was filed on Aug. 24, 2020, which claims priority to European Patent Application No. 19193680.6, which was filed on Aug. 27, 2019. The contents of each are hereby incorporated by reference into this specification.

### FIELD

The present invention relates to a process for preparing bisphenol A, a process for preparing polycarbonate and a composition comprising bisphenol A and at least one specific impurity which is formed in the production of bisphenol A.

### BACKGROUND

Bisphenol A or BPA is an important monomer in the production of polycarbonate or epoxy resins. Normally, BPA is used in the form of para,para-BPA (2,2-Bis(4-hydroxyphenol)propane; p,p-BPA). However, in the production of BPA also ortho,ortho-BPA (o,o-BPA) and/or ortho,para-BPA (o,p-BPA) may be formed. In principle, when referring to BPA, reference is made to para,para-BPA still containing low amounts of ortho,ortho-BPA and/or ortho,para-BPA.

According to the state of the art BPA is produced by reacting phenol with acetone in the presence of an acid catalyst to give the bisphenol. In former times hydrochloric acid (HCl) was used for the commercial process of the condensation reaction. Today, a heterogeneous continuous process for the production of BPA is used in the presence of an ion exchange resin catalyst, wherein said ion exchange resin comprises a crosslinked acid functionalized polystyrene resin. The most important resins are crosslinked polystyrenes with sulfonic acid groups. Divinylbenzene is mostly used as the crosslinking agent as described in GB849965, U.S. Pat. No. 4,427,793, EP0007791 and EP0621252 or Chemistry and properties of crosslinked polymers, edited by Santokh S. Labana, Academic Press, New York 1977.

In order to achieve a high selectivity, the reaction of phenol with acetone can be performed in the presence of suitable co-catalyst. It is known that the catalyst deactivates over time. For example, the deactivation is described in EP0583712, EP10620041, DE14312038. One major objective for the production process is to maximize the performance and dwell times of the catalyst system. Accordingly, there is a need to identify potential poisonous substances, by-products, impurities of educts etc. in order to deal with this objective.

U.S. Pat. No. 5,414,151 A teaches that improved bisphenol production and an extension in the life of the bisphenol condensation catalyst can be achieved by using as the phenol reactant, a material having less than about 1 ppm of hydroxyacetone. Here the catalyst system comprises an ion exchange resin catalyst and a sulfur containing cocatalyst, wherein the co-catalyst is chemically bound to the ion exchange resin catalyst

WO2012/150560 A1 teaches the use of a specific catalyst system comprising an ion exchange resin catalyst and a sulfur containing cocatalyst, wherein the co-catalyst is chemically bound to the ion exchange resin catalyst, and also a process for catalyzing condensation reactions between phenols and ketones using such specific catalyst system. Furthermore WO2012/150560 A1 discloses a process for catalyzing condensation reactions between phenols and ketones that does not utilize a bulk promoter that is not chemically bound to the ion exchange resin catalyst.

Thus, the prior art clearly states that a catalyst system comprising an ion exchange resin catalyst and a chemically bound sulfur containing cocatalyst is prone to hydroxyacetone poisoning. Accordingly, the prior art teaches that the concentration of hydroxyacetone as impurity in raw phenol and raw acetone needs to be reduced as low as possible in order to avoid catalyst poisoning.

However, the removal of hydroxyacetone from raw phenol and/or raw acetone consumes time and money and thus, renders the raw phenol and/or raw acetone more expensive. In the end it increases the costs of bisphenol A and the respective polymer prepared from this bisphenol A. Moreover, the concentration of hydroxyacetone in raw phenol and/or raw acetone varies depending on the supplier and their process of purification of these raw materials. This means that different raw material qualities need to be handled (e. g. another step of purification needs to be performed if the specification exceeds a certain threshold) decreasing flexibility of the process and choice of raw material supplier.

### SUMMARY

Therefore, it was an object of the present invention to provide a process for the preparation of ortho,para-, ortho,ortho- and/or para,para-bisphenol A via condensation of phenol and acetone which is more economical than the processes of the prior art. Moreover, it was an object of the present invention to provide a process for the preparation of ortho,para-, ortho,ortho- and/or para,para-bisphenol A via condensation of phenol and acetone which is more flexible and/or which allows more flexibility in the choice of the quality of raw phenol and/or raw acetone. This flexibility should be preferably provided with respect to the concentration of hydroxyacetone as impurity in raw phenol and/or raw acetone.

At least one of the above-mentioned objects, preferably all of these objects have been solved by the present invention. Surprisingly, it has been found that a catalyst system comprising an ion exchange resin catalyst and a sulfur containing cocatalyst, wherein at least part of the sulfur containing cocatalyst is not chemically bound to the ion exchange resin catalyst, is not susceptible to catalyst poisoning by hydroxyacetone. This is surprising, because the prior art suggests that a catalyst system comprising a chemically bound sulfur containing cocatalyst is prone to such poisoning. Moreover, the prior art teaches the necessity to reduce the amount of hydroxyacetone in raw acetone and/or raw phenol as low as possible. Due to the fact that the specific catalyst system of the present invention is not affected by this impurity, cheaper raw acetone and/or raw phenol can be used without the risk of reducing catalyst life time. This renders the overall process more cost effective. In addition, as less energy for purifying the raw materials is needed, the process becomes more ecologically advantageous. Moreover, the process allows more flexibility in the choice of the quality of raw phenol and/or raw acetone, especially with respect to the concentration of hydroxyacetone in those raw materials.

Accordingly, the present invention provides a process for preparing ortho,para-, ortho,ortho- and/or para,para-bisphenol A comprising the step of
(a) condensing raw phenol and raw acetone in the presence of a catalyst system, wherein the catalyst system comprises an ion exchange resin catalyst and a sulfur containing cocatalyst, wherein at least part, preferably 75 mol-% of the sulfur containing cocatalyst is not chemically bound to the ion exchange resin catalyst,
   characterized in that the amount of hydroxyacetone present in step (a) is higher than 1.2 ppm with respect to the total weight of the sum of the weights of the raw phenol and the raw acetone.

### DETAILED DESCRIPTION

According to the present invention reference is made to “raw phenol” and/or “raw acetone”. The term “raw” is used for the unreacted educts as applied, especially added, in the process for preparing BPA. In particular, this term is used to distinguish the phenol which is freshly added to the reaction (as raw phenol) and the phenol which is recycled in the process for preparing BPA (recycled phenol). Such recycled phenol cannot add additional hydroxyactone to the process. The same holds true for acetone which is freshly added to the reaction (as raw acetone) and acetone which is recycled in the process for preparing BPA (recycled acetone). When referring to phenol and/or acetone without any further specification it is preferred that the sum either the chemical compound as such or both raw and recycled phenol and/or raw and recycled acetone are meant.

Hydroxyacetone is an impurity in both raw materials of the reaction of BPA. Raw phenol and raw acetone can both contain hydroxyacetone impurities. For example, the production pathways for aceton or phenol are described in Arpe, Hans-Jürgen, Industrielle Organische Chemie, 6. Auflage, January 2007, Wiley-VCH. In particular the process for preparing phenol is described in Ullmann's Encyclopedia of Industrial Chemistry, chapters Phenol and Phenol derivatives. The oxidation of cumene, also known as Hock process, is by far the dominant synthetic route to phenol. Among the contaminants formed during the manufacture of phenol are hydroxyketones, especially hydroxyacetone.

The process of the present invention is characterized in that the amount of hydroxyacetone present in step (a) is higher than 1.2 ppm, preferably higher than 1.3 ppm, more preferably higher than 1.4 ppm, still more preferably higher than 1.5 ppm, still preferably higher than 2 ppm, still more preferably higher than 5 ppm, still more preferably higher than 10 ppm and most preferably higher than 50 ppm with respect to the total weight of the sum of the weights of the raw phenol and the raw acetone. Moreover, it is preferable that the amount of hydroxyacetone present in step (a) is higher than 1.2 ppm and equal to or lower than 5000 ppm, more preferably equal to or lower 4500 ppm, still more preferably equal to or lower 4000 ppm, still more preferably equal to or lower 3500 ppm, still more preferably equal to or lower 3000 ppm, still more preferably equal to or lower 2500 ppm and most preferably equal to or lower 2000 ppm with respect to the total weights of the raw phenol and the raw acetone. The skilled person knows how to determine the amount of hydroxyacetone in raw phenol and/or raw acetone. For example, the amount of hydroxyacetone in raw phenol can be determined according to ASTM D6142-12 (2013). The amount of hydroxyacetone in raw acetone can be determined by gas chromatography. For example, formerly the purity of acetone has been determined by ASTM D1154 which is now withdrawn.

According to the present invention “ppm” preferably refers to parts by weight.

Preferably, the process of the present invention is characterized in that the process additionally comprises the following step:

(b) separating the mixture obtained after step (a) into a bisphenol A fraction comprising at least one of ortho,para-, ortho,ortho- and/or para,para-bisphenol A and a phenol fraction, wherein the phenol fraction comprises unreacted phenol and at least one impurity formed due to the presence of hydroxyacetone in step (a).

Preferably, the bisphenol A fraction is taken as product and/or further purified. Several variants of production processes exist to provide the bisphenol of high purity. This high purity is especially of importance for the use of BPA as monomer in the production of poly carbonates. WO-A 0172677 describes crystals of an adduct of a bisphenol and of a phenol and a method for producing these crystals and finally preparing bisphenols. It was found that by crystallizing these adducts a para,para-BPA of high purity can be obtained. EP1944284 describes the process for producing bisphenols wherein the crystallization comprises continuous suspension crystallization devices. It is mentioned that the requirements with respect to the BPA purity are increasing and that with the disclosed method a very pure BPA of higher than 99.7% can be obtained. WO-A 2005075397 describes a process for producing bisphenol A in which the water that is produced during the reaction is removed by distillation. By this method the unreacted acetone is recovered and recycled resulting in an economically favorable process.

Preferably, the process of the present invention is characterized in that the separation in step (b) is performed using a crystallization technique. Still preferably, the separation in step (b) is performed using at least one continuous suspension crystallization device.

It has been further described to make use of a mother liquor cycle. BPA is taken out of the solvent by crystallization and filtration after the reaction. The mother liquor typically contains 5 to 20% BPA and byproducts dissolved in unreacted phenol. Moreover, water is formed during the reaction and is removed from the mother liquor in the dewatering section. Preferably, the fraction comprising unreacted phenol is recycled for further reaction. This preferably means that the mother liquor is recycled. It is re-used as unreacted phenol in the reaction with acetone in order to give BPA. The flow of mother liquor is preferably conventionally recirculated into the reaction unit.

Typically byproducts in the mother liquor are for example o,p-BPA, o,o-BPA, substituted indenes, hydroxyphenyl indanoles, hydroxyphenyl chromanes, substituted xanthenes and higher condensed compounds. In addition, further secondary compounds such as anisole, mesityl oxide, mesitylene and diaceton alcohol may be formed as a result of self-condensation of the acetone and reaction with impurities in the raw material.

Due to the recycling of mother liquor byproducts accumulate in the circulation stream and can lead to an additional deactivation of the catalyst system. This means for a prolonged use of a catalyst, the impact of initial impurities in the educts have to be considered as well as the impact of possible byproducts in the reaction itself, resulting either from the reaction of phenol with acetone or from a reaction of one of the impurities.

In a further aspect of the present invention it has been found that the presence of hydroxyacetone in process step (a) (reaction of phenol with acetone) leads to the formation of new by-products or impurities. It has been found that the hydroxyacetone reacts during the process of the present invention and cannot be detected any longer in subsequent process steps. Accordingly, preferably the process of the present invention is characterized in that after performing step (a), the amount of hydroxyacetone in the mixture resulting from step (a) is lower than 1 ppm, preferably 0.00001 to 0.9 ppm, still preferably 0.0001 to 0.5 ppm and most preferably 0.001 to 0.1 ppm with respect to the total weight of the mixture resulting from step (a). However, a new compound which will be describe below has been identified which seems to have been formed due to the presence of hydroxyacetone in step (a).

Accordingly, it is preferred that the process of the present invention is characterized in that the process comprises the additional step of

(c) using at least a part of the phenol fraction obtained in step (b) as educt in step (a), wherein the part of the phenol fraction comprises not more than 1 ppm, preferably 0.00001 to 0.9 ppm, still preferably 0.0001 to 0.5 ppm and most preferably 0.001 to 0.1 ppm of hydroxyacetone with respect to the total weight of the phenol fraction.

In order to avoid accumulation of the byproducts and/or impurities formed due to the presence of hydroxyacetone in step (a) in the system several options exist. One is a purge stream, for example a portion of the mother liquor is discharged. Another approach comprises the passing a part of the entire amount of the circulation stream after solid/liquid separation and before or after the removal of water and residual acetone, over e. g. a rearrangement unit filled with acid ion exchanger. In this rearrangement unit some of the byproducts from BPA preparation are isomerized to give p,p-BPA. It has been found that the new impurity which forms due to the presence of hydroxyacetone in process step (a) can be removed by a purge stream. Accordingly, it has preferred that at least part of the phenol fraction obtained in step (b) is used as educt in step (a), wherein at least a part of this stream is purged. Preferably, more than 50 vol.-% of the phenol fraction obtained in step (b) is used as educt in step (a), wherein the vol.-% is based on the total volume of the phenol fraction.

Preferably, the process of the present invention is characterized in that the at least one impurity formed due to the presence of hydroxyacetone in step (a) is selected from the group consisting of 4-(2,2,4-trimethyl-4-chromanyl)phenol, 2,4,4-trimethyl-2-(4-hydroxyphenyl)chromane, compound M362, compound M434 and mixtures thereof, wherein compound M362 is a compound having a molecular weight of 362 g/mol, three OH groups and a retention time of 25.37 seconds in a gas chromatography and M434 is a compound having a molecular weight of 434 g/mol, two OH groups and a retention time of 25.37 seconds in a gas chromatography, wherein the gas chromatography is coupled to mass spectroscopy for identification of M362/M434 using a column from Agilent J&W VF-1MS (100% Dimethylpolysiloxane) of the size of 25 m×0.2 mm×0.33 μm, a temperature profile of 80° C. for 0.10 min, heating with 10° C./min to 280° C. and holding this temperature for 10.00 min; injecting 1 μl with a split of 10/1 at 250° C.); wherein the flow is 1 ml/min at an initial pressure of 24.45 psi (1.685768 bar) and the mass spectrometer scans from mz35 -mz 700.

According to the present invention it has been found that hydroxyacetone leads to the formation of chromanes and a molecule of higher molecular weight. The structure of this compound is unknown and the molecular weight can be either 362 g/mol or 434 g/mol. Those compounds are referred to as M362 and M434. Although the exact structure of M362 and M434 is not known, but they can be easily and reproducibly detected using the gas chromatography analysis as described above and in the examples. For the analysis the compound is silylated. Depending on whether this compound either has three or two OH groups which can be silylated the molecular weight is either 362 g/mol or 434 g/mol.

This gas chromatography is coupled with mass spectroscopy for identification of M362 and M434 is performed as described above.

Compound M362 or M434 is inter alia defined by a retention time which is determined by gas chromatography. This retention time is given quite exactly. However, the skilled person knows that minor variations can occur even in case the exact method as given with respect to this invention is followed. Therefore, those variation are encompassed according to the present invention as long as the signal can be clearly attributed to the specific compound.

Still preferably, the process of the present invention is characterized in that in step (a) compound M362 or compound M434 is present, wherein compound M362 is a compound having a molecular weight of 362 g/mol, three OH groups and a retention time of 25.37 seconds in a gas chromatography and M434 is a compound having a molecular weight of 434 g/mol, two OH groups and a retention time of 25.37 seconds in a gas chromatography, wherein the gas chromatography is coupled by mass spectroscopy as described above. This means that due to the presence of hydroxyacetone in process step (a) a compound M362 or M434 needs to be mandatorily present in process step (a), too, because hydroxyacetone forms those impurities. However, because no deactivation of the catalyst has been observed, these impurities do not seem to poison the catalyst at least at small amounts. Moreover, those impurities can be present in process step (a) in case the phenol fraction of step (b) is recycled in process step (c). The accumulation of these impurities can be preferably avoided by using a purge stream as described above.

The catalyst system which can be used in the process of the present invention is known by the skilled person. Preferably, it is an acidic ion exchange resin. Such ion exchange resin can have from 2% to 20%, preferably 3 to 10% and most preferably 3.5 to 5.5% crosslinkage. The acidic ion exchange resin preferably can be selected from the group consisting of sulfonated styrene divinyl benzene resins, sulfonated styrene resins, phenol formaldehyde sulfonic acid resins and benzene formaldehyde sulfonic acid. Moreover, the ion exchange resin may contain sulfonic acid groups. The catalyst bed can be either a fixed bed or a fluidized bed.

Furthermore, the catalyst system of the present invention comprises a sulfur containing cocatalyst, wherein at least part of the sulfur containing cocatalyst is not chemically bound to the ion exchange resin catalyst. The sulfur containing cocatalyst can be one substance or a mixture of at least two substances. This cocatalyst is preferably dissolved in the reaction solution of process step (a). Still preferably, the cocatalyst is dissolved homogenously in the reaction solution of process step (a). Preferably, the process of the present invention is characterized in that the sulfur containing cocatalyst is selected from the group consisting of mercaptopropionic acid, hydrogen sulfide, alkyl sulfides such as ethyl sulfide and mixtures thereof. Most preferably, the sulfur containing cocatalyst is 3-mercaptopropionic acid.

Preferably, the catalyst system of the present invention comprises a sulfur containing cocatalyst, wherein all of the sulfur containing cocatalyst is not chemically bound to the ion exchange resin catalyst. This means that preferably all of the sulfur containing cocatalyst is added to the process step (a). According to the present invention the expression “not chemically bound” refers to a catalyst system where neither a covalent nor an ionic bound between the ion exchange resin catalyst and the sulfur containing cocatalyst is present at the beginning of process step (a). However, this does not mean that at least part of the sulfur containing cocatalyst might get fixed to the heterogeneous catalyst matrix via ionic or covalent bonds. Nevertheless, at the beginning of process step (a) no such ionic or covalent bonds of the sulfur containing cocatalyst are present, but if they are formed at all, they are formed over time. Accordingly, preferably the sulfur containing cocatalyst is added to process step (a). The term “added” describes an active process step. This means, as said above, that the cocatalyst is preferably dissolved in the reaction solution of process step (a). Additionally the cocatalyst can be added at any other process step or even twice or more times at process step (a). Moreover, preferably, most of the sulfur containing cocatalyst is not chemically bound to the ion exchange resin catalyst. This means that at least 75 mol-%, still preferably at least 80 mol-%, most preferably at least 90 mol-% of the sulfur containing cocatalyst is not chemically bound to the ion exchange resin catalyst. Here the mol-% relate to the total sum of the cocatalyst present in process step (a).

Because hydroxyacetone is a common impurity in raw phenol and raw acetone, it is preferred that the hydroxyacetone present in step (a) is introduced into the process step (a) as impurity in the raw acetone and/or the raw phenol. Nevertheless, at least part of the hydroxyacetone can be present in process step (a) due to other reasons.

In another aspect the present invention provides a process for preparing polycarbonate comprising the steps of
(i) obtaining a ortho,para-, ortho,ortho- and/or para,para-bisphenol A according to the process of the present invention in any embodiment or combination of preferred embodiments and
(ii) polymerizing the ortho,para-, ortho,ortho- and/or para,para-bisphenol A obtained in step (i), optionally in the presence of at least one further monomer in order to obtain a polycarbonate.

As explained above, the process for the production of ortho,para-, ortho,ortho- and/or para,para-bisphenol A of the present invention provides a BPA which can be obtained in a more economical and/or ecological way. Accordingly, in using this BPA as obtained with the process according to the present invention, the process for preparing polycarbonate according to the present invention is more economical and/or ecological, too.

Reaction step (ii) is known to the skilled person. The polycarbonates can be prepared in a known manner from the BPA, carbonic acid derivatives, optionally chain terminators and optionally branching agents by interphase phosgenation or melt transesterification.

In the interphase phosgenation bisphenols and optionally branching agents are dissolved in aqueous alkaline solution and reacted with a carbonate source, such as phosgene, optionally dissolved in a solvent, in a two-phase mixture comprising an aqueous alkaline solution, an organic solvent and a catalyst, preferably an amine compound. The reaction procedure can also be effected in a plurality of stages. Such processes for the preparation of polycarbonate are known in principle as interfacial processes, for example from H. Schnell, Chemistry and Physics of Polycarbonates, Polymer Reviews, Vol. 9, Interscience Publishers, New York 1964 page 33 et seq., and on Polymer Reviews, Vol. 10, “Condensation Polymers by Interfacial and Solution Methods”, Paul W. Morgan, Interscience Publishers, New York 1965, chapter VIII, page 325, and the underlying conditions are therefore familiar to the person skilled in the art.

Alternatively, polycarbonates may also be prepared by the melt transesterification process. The melt transesterification process is described, for example, in Encyclopaedia of Polymer Science, Vol. 10 (1969), Chemistry and Physics of Polycarbonates, Polymer Reviews, H. Schnell, Vol, 9, John Wiley and Sons, Inc. (1964), and DE-C 10 31 512. In the melt transesterification process, the aromatic dihydroxy compounds already described in the case of an interfacial process are transesterified with carbonic acid diesters with the aid of suitable catalysts and optionally further additives in the melt.

Preferably, the process for preparing polycarbonates according to the present invention is characterized in that the process step (i) further comprises a step of purifying the ortho,para-, ortho,ortho- and/or para,para-bisphenol A in order to reduce the amount of at least one impurity formed due to the presence of hydroxyacetone in step (a). As has been described above, cheaper raw phenol and/or raw acetone can be used in the process of the present invention. However, when having hydroxyacetone as impurity in these cheaper raw materials, other impurities are formed. These impurities are preferably removed before the polymerization.

Still preferably, the process for preparing polycarbonates according to the present invention is characterized in that the at least one impurity formed due to the presence of hydroxyacetone in step (a) is selected from the group consisting of ortho,para-bisphenol A, 4-(2,2,4-trimethyl-4-chromanyl)phenol, 2,4,4-trimethyl-2-(4-hydroxyphenyl)chromane, compound M362, compound M434 and mixtures thereof, wherein compound M362 is a compound having a molecular weight of 362 g/mol, three OH groups and a retention time of 25.37 seconds in a gas chromatography and M434 is a compound having a molecular weight of 434 g/mol, two OH groups and a retention time of 25.37 in a gas chromatography, wherein the gas chromatography is coupled by mass spectroscopy as described above.

In still another aspect of the present invention a composition is provided comprising ortho,para-, ortho,ortho- and/or para,para-bisphenol A and compound M362 or compound M434, wherein compound M362 is a compound having a molecular weight of 362 g/mol, three OH groups and a retention time of 25.37 seconds in a gas chromatography and M434 is a compound having a molecular weight of 434 g/mol, two OH groups and a retention time of 25.37 in a gas chromatography, wherein the gas chromatography is coupled by mass spectroscopy as described above.

Moreover, this composition of the present invention is preferably further characterized in that the composition comprises less than 1 ppm, preferably 0.00001 to 0.9 ppm, still preferably 0.0001 to 0.5 ppm and most preferably 0.001 to 0.1 ppm of hydroxyacetone with respect to the total weight of the composition.

### EXAMPLES

Materials used in the examples:

| | |
|---|---|
| catalyst | acidic polymer-based resin (polystyrene-divinyl- |
| | benzene) with sulfonic acid groups, spherical |
| | beads, 4% of crosslinking, |
| phenol | Acros Organics, grade: extra pure > 99 % |
| | (measured purity by GC: 99.9%) |
| MEPA | 3-mercaptopropionic acid, Sigma-Aldrich, |
| | Purity > 99 % (measured purity by GC: 99.3%) |
| acetone | VWR, GPR rectapur > 99.5 % (measured purity |
| | by GC: 100%, water not included) |
| hydroxyacetone | Sigma Aldrich, purity 99.0 % |
| methanol | Fisher Analytical grade; GC purity 99.99% |

A column reactor was equipped with 150 g of the phenol-wet catalyst (volume of phenol-wet catalyst in the reactor: 210 to 230 ml). The column reactor was heated to 60° C. (catalyst bed temperature during reaction: 63° C.). A mixture of phenol, acetone (3.9 wt.-%) and MEPA (160 ppm with respect to the sum of the masses of phenol and acetone) was prepared and tempered to 60° C. This mixture was pumped into the column reactor with a flow rate of 45 g/h. The column reactor was equipped with a sampling point at the bottom. Using the aperture of the sampling point, different samples were taken during the reaction. Sampling time was 1 h and the amount of the sample taken each hour was 45 g.

A first run (standard run) was conducted for 52 h. After 48 h, 49 h, 50 h and 51 h, respectively, a sample was taken and analyzed via GC.

A second run (impurity run) was conducted for 52 h. At the beginning of the second run 2200 ppm (with respect to the sum of the masses of phenol and acetone) of hydroxyacetone was dosed to the reaction system. After 48 h, 49 h, 50 h and 51 h, respectively, a sample was taken and analyzed via GC. After this a fresh mixture of acetone, phenol and MEPA was used and a third run (standard run) was conducted for 52 h. After 48 h, 49 h, 50 h and 51 h, respectively, a sample was taken via a syringe and analyzed via GC. Then a fourth run (impurity run) was conducted for 52 h. At the beginning of the fourth run 2200 ppm (with respect to the sum of the masses of phenol and acetone) of hydroxyacetone was dosed to the reaction system. After 48 h, 49 h, 50 h and 51 h, respectively, a sample was taken and analyzed via GC. Finally, a fifth run (standard run) was conducted for 52 h. After 48 h, 49 h, 50 h and 51 h, respectively, a sample was taken and analyzed via GC.

The gaschromatography (GC) for methanol was conducted using a column Agilent J&W VF-1MS (100% Dimethylpolysiloxane) of the size 50 m×0.25 mm×0.25 μm, a temperature profile of 60° C. for 0.10 min, heating with 12° C./min to 320° C. and holding this temperature for 10.00 min; injecting 1 μl with a split of 10/1 at 300° C.); wherein the flow is 2 ml/min at an initial pressure of 18.3 psi (1.26 bar)

The gaschromatography (GC) for hydroxyacetone, phenol, para,para BPA were conducted using a column Agilent J&W VF-1MS (100% Dimethylpolysiloxane) of the size 50 m×0.25 mm×0.25 μm, a temperature profile of 80° C. for 0.10 min, heating with 12° C./min to 320° C. and holding this temperature for 10.00 min; injecting 1 μl with a split of 10/1 at 300° C.); wherein the flow is 2 ml/min at an initial pressure of 18.3 psi (1.26 bar)

The gas chromatography (GC) coupled to mass spectroscopy (MS) for identification of M362/M434 is performed using a column Agilent J&W VF-1MS (100% Dimethylpolysiloxane) of the size 25 m×0.2 mm×0.33 μm, a temperature profile of 80° C. for 0.10 min, heating with 10° C./min to 280° C. and holding this temperature for 10.00 min; injecting 1 μl with a split of 10/1 at 250° C.); wherein the flow is 1 ml/min at an initial pressure of 24.45 psi (1.685768 bar) and the mass spectrometer scans from mz35-mz 700

The standard run represents the reaction of acetone and phenol in the presence of the catalyst and cocatalyst to form BPA. From this the acetone conversion can be estimated including respective error bars. This conversion represented the baseline to evaluate whether the impurities influence the catalyst deactivation or not. The acetone conversion of standard runs 3 and 5 were compared to the value of standard run 1 to determine the effect of hydroxyacetone on the catalyst. If the acetone conversion dropped out of this conversion, it would be proven that hydroxyacetone has an effect on the BPA catalyst. In order to show that this kind of evaluation can be used to determine catalyst poisoning, a reference run was conducted using methanol as impurity. It is known from the state of the art that methanol is a known poison for the catalyst in the BPA process that is described for example in U.S. Pat. No. 8,143,456. Table 1 shows the respectively obtained results. The values given in the table are the average values obtained from the four samples taken during each run (after 48 h, 49 h, 50 h and 51 h).

TABLE 1
reference run with methanol

| | | | second | | | |
|---|---|---|---|---|---|---|
| | | first run | run | third run | fourth run | fifth run |
| | | (standard | (impurity | (standard | (impurity | (standard |
| Substance | Unit | run) | run) | run) | run) | run) |
| Acetone | % | 82.63 | 78.65 | 81.92 | 78.20 | 79.42 |
| conversion | | | | | | |
| Methanol IN** | mg/kg | — | 1710 | — | 1660 | — |

**The amount of methanol IN is measured before the catalyst.

As can be clearly seen from table 1, the acetone conversion of each standard run 1, 3 and 5 drops. This means that the catalyst is poisoned by methanol and the conversion cannot be recovered due to irreversible reactions which decrease the catalyst activity.

The following table shows the results of the first run (standard run), the second run (impurity run), the third run (standard run), the fourth run (impurity run) and the fifth run (standard run) for hydroxyacetone as impurity. The values given in the table are the average values obtained from the four samples taken during each run (after 48 h, 49 h, 50 h and 51 h).

TABLE 2
Hydroxy acetone

| | | | second | | | |
|---|---|---|---|---|---|---|
| | | first run | run | third run | fourth run | fifth run |
| | | (standard | (impurity | (standard | (impurity | (standard |
| Substance | Unit | run) | run) | run) | run) | run) |
| Acetone | % | 81.99 | 73.16 | 82.88 | 73.14 | 82.24 |
| conversion | | | | | | |
| Hydroxyacetone | mg/kg | — | 2200 | — | 2200 | — |
| IN** | | | | | | |
| Hydroxyacetone | mg/kg | — | <5 | — | <5 | — |
| OUT** | | | | | | |
| M362/M343 | mg/kg | <5 | <5 | <5 | <5 | <5 |

**The amount of hydroxyacetone IN is measured before the catalyst. The amount of hydroxyacetone OUT is measured from the four samples taken during each run (after 48 h, 49 h, 50 h and 51 h; average value).

As can be seen from the results of table 2, the addition of hydroxyacetone in a reaction of phenol and acetone to para,para-BPA leads to no drop in acetone conversion for the standard runs 1, 3 and 5. This means that hydroxyacetone is no poison for the catalyst system used. This effect can be seen after each impurity run. Moreover, it can be seen that nearly all hydroxyacetone reacts during the impurity runs (no hydroxyacetone OUT can be detected).

1. A process for preparing ortho,para-, ortho,ortho- and/or para,para-bisphenol A comprising the step of
(a) condensing raw phenol and raw acetone in the presence of a catalyst system, wherein the catalyst system comprises an ion exchange resin catalyst and a sulfur containing cocatalyst, wherein at least 75 mol-% of the sulfur containing cocatalyst is not chemically bound to the ion exchange resin catalyst,
wherein the amount of hydroxyacetone present in step (a) is higher than 1.2 ppm with respect to the total weight of the sum of the weights of the raw phenol and the raw acetone.

2. The process according to claim 1, wherein the amount of hydroxyacetone present in step (a) is higher than 1.2 ppm and equal to or lower than 5000 ppm with respect to the total weights of the raw phenol and the raw acetone.

3. The process according to claim 1, wherein the process additionally comprises the following step:
(b) separating the mixture obtained after step (a) into a bisphenol A fraction comprising at least one of ortho,para-, ortho,ortho- and/or para,para-bisphenol A and a phenol fraction, wherein the phenol fraction comprises unreacted phenol and at least one impurity formed due to the presence of hydroxyacetone in step (a).

4. The process according to claim 3, wherein the separation in step (b) is performed using a crystallization technique.

5. The process according to claim 3, wherein the process comprises the additional step of
(c) using at least a part of the phenol fraction obtained in step (b) as educt in step (a), wherein the part of the phenol fraction comprises not more than 1 ppm of hydroxyacetone with respect to the total weight of the phenol fraction.

6. The process according to claim 3, wherein the at least one impurity formed due to the presence of hydroxyacetone in step (a) is selected from the group consisting of 4-(2,2,4-trimethyl-4-chromanyl)phenol, 2,4,4-trimethyl-2-(4-hydroxyphenyl)chromane, compound M362, compound M434 and mixtures thereof, wherein compound M362 is a compound having a molecular weight of 362 g/mol, three OH groups and a retention time of 25.37 seconds in a gas chromatography and M434 is a compound having a molecular weight of 434 g/mol, two OH groups and a retention time of 25.37 seconds in a gas chromatography, wherein the gas chromatography is performed using a column Agilent J&W VF-1MS of the size 25 m×0.2 mm×0.33 μm; a temperature profile of 80° C. for 0.10 min, heating with 10° C./min to 280° C. and holding this temperature for 10.00 min; injecting 1 μl with a split of 10/1 at 250° C.; wherein the flow is 1 ml/min at an initial pressure of 24.45 psi (1.685768 bar) and the mass spectrometer scans from mz35 to mz 700.

7. The process according to claim 1, wherein after performing step (a), the amount of hydroxyacetone in the mixture resulting from step (a) is lower than 1 ppm with respect to the total weight of the mixture resulting from step (a).

8. The process according to claim 1, wherein in step (a) compound M362 or compound M434 is present, wherein compound M362 is a compound having a molecular weight of 362 g/mol, three OH groups and a retention time of 25.37 seconds in a gas chromatography and M434 is a compound having a molecular weight of 434 g/mol, two OH groups and a retention time of 25.37 seconds in a gas chromatography, wherein the gas chromatography is performed using a column Agilent J&W VF-1MS of the size 25 m×0.2 mm×0.33μm; a temperature profile of 80° C. for 0.10 min, heating with 10° C./min to 280° C. and holding this temperature for 10.00 min; injecting 1 μl with a split of 10/1 at 250° C.; wherein the flow is 1 ml/min at an initial pressure of 24.45 psi (1.685768 bar) and the mass spectrometer scans from mz35 to mz 700.

9. The process according to claim 1, wherein the sulfur containing cocatalyst is selected from the group consisting of mercaptopropionic acid, hydrogen sulfide, alkyl sulfides such as ethyl sulfide and mixtures thereof.

10. The process according to claim 1, wherein the hydroxyactone present in step (a) is introduced into the process step (a) as impurity in the raw acetone and/or the raw phenol.

11. A process for preparing polycarbonate comprising the steps of
(i) obtaining a ortho,para-, ortho,ortho- and/or para,para-bisphenol A according to the process of claim 1 and
(ii) polymerizing the ortho,para-, ortho,ortho- and/or para,para-bisphenol A obtained in step (i).

12. The process according to claim 11, wherein the process step (i) further comprises a step of purifying the ortho,para-, ortho,ortho- and/or para,para-bisphenol A in order to reduce the amount of at least one impurity formed due to the presence of hydroxyacetone in step (a).

13. The process according to claim 12, wherein the at least one impurity formed due to the presence of hydroxyacetone in step (a) is selected from the group consisting of ortho,para-bisphenol A, 4-(2,2,4-trimethyl-4-chromanyl)phenol, 2,4,4-trimethyl-2-(4-hydroxyphenyl)chromane, compound M362, compound M434 and mixtures thereof, wherein compound M362 is a compound having a molecular weight of 362 g/mol, three OH groups and a retention time of 25.37seconds in a gas chromatography and M434 is a compound having a molecular weight of 434 g/mol, two OH groups and a retention time of 25.37 seconds in a gas chromatography, wherein the gas chromatography is coupled to mass spectroscopy for identification of M362/M434 using a column Agilent J&W VF-1MS (100% Dimethylpolysiloxane) of the size 25 m×0.2 mm×0.33 μm, a temperature profile of 80° C. for 0.10 min, heating with 10° C./min to 280° C. and holding this temperature for 10.00 min; injecting 1 μl with a split of 10/1 at 250° C.); wherein the flow is 1 ml/min at an initial pressure of 24.45 psi (1.685768 bar) and the mass spectrometer scans from mz35-mz 700.

14. A composition comprising ortho,para-, ortho,ortho- and/or para,para-bisphenol A and compound M362 or compound M434, wherein compound M362 is a compound having a molecular weight of 362 g/mol, three OH groups and a retention time of 25.37 seconds in a gas chromatography and M434 is a compound having a molecular weight of 434 g/mol, two OH groups and a retention time of 25.37 seconds in a gas chromatography wherein the gas chromatography is gas chromatography is coupled to mass spectroscopy for identification of M362/M434 using a column Agilent J&W VF-1MS (100% Dimethylpolysiloxane) of the size 25 m×0.2 mm×0.33 μm, a temperature profile of 80° C. for 0.10 min, heating with 10° C./min to 280° C. and holding this temperature for 10.00 min; injecting 1 μl with a split of 10/1 at 250° C.); wherein the flow is 1 ml/min at an initial pressure of 24.45 psi (1.685768 bar) and the mass spectrometer scans from mz35-mz 700.

15. The composition according to claim 14, wherein the composition comprises less than 1 ppm hydroxyacetone with respect to the total weight of the composition.

16. The process according to claim 11, wherein the polymerization of step (ii) is performed in the presence of at least one further monomer in order to obtain a polycarbonate.
